(19) United States
(12) Patent Application Publication
(10) Pub. No.: US 2012/0302487 A1
(43) Pub. Date: Nov. 29, 2012

(54) COMPOSITE

(75) Inventors: Stephen Armstrong, Waverton (GB); Terence Cosgrove, Portishead (GB); John Jeffrey Gerrard, Chester (GB); Melaine Jane Hughes, Chester (GB); David Alan Pears, Poynton (GB)

(73) Assignee: REVOLYMER LIMITED, London (GB)

(21) Appl. No.: 13/504,735

(22) PCT Filed: Oct. 28, 2010
(86) PCT No.: PCT/GB2010/002007

§ 371 Date: Aug. 1, 2012

(30) Foreign Application Priority Data
Oct. 28, 2009 (GB) 0918914.3

(51) Int. Cl. C11D 17/00; B05D 7/00; C11D 7/54; C08F 20/06; C07C 407/00; C11D 3/60

(52) U.S. Cl. 510/313; 562/4; 510/513; 510/349; 510/312; 252/186.25; 526/317.1; 427/212; 427/213

(57) ABSTRACT

A composite includes: one or more core units comprising a bleach activating agent; and an alkali soluble polymer coating on the surface of said one or more core units.

The present invention relates to a composite comprising a bleach activating agent and a polymeric coating, and use thereof in the field of detergents, particularly liquid detergents.

### BACKGROUND TO THE INVENTION

It is well established in the field of detergents that certain sensitive constituents, such as bleach components, must be protected from an incompatible environment by physical separation, for example, by encapsulation.

Tetraacetylethylenediamine (TAED) is one example of such a component. TAED is included in bleach boosters and laundry soak treatments (to improve wash performance) as well as being used in the bleaching of wood pulp and textiles. However, the most significant commercial application of tetraacetylethylenediamine is in powdered laundry detergents, where it is used as a bleach activator (or bleach precursor) for active oxygen bleaching agents such as sodium percarbonate, sodium perborate, sodium perphosphate and sodium persulphate, which release hydrogen peroxide during the wash cycle. The behaviour of such active oxygen bleaching agents is extremely temperature sensitive. Moreover, hydrogen peroxide is an inefficient bleaching agent below 40° C. Thus, in the absence of a bleach activator, wash temperatures of greater than 60° C. are typically required in order to achieve effective stain removal. However, such high wash temperatures are economically and practically disadvantageous. This problem is addressed by the use of bleach activators (commonly esters or amides of carboxylic acids such as tetraacetylethylenediamine), that react with hydrogen peroxide to generate a peracid.

In the case of tetraacetylethylenediamine, peracetic acid (CH₃CO₃H) is generated by the reaction of hydrogen peroxide with tetraacetylethylenediamine (1) and then triacetylethylenediamine (2) to yield diacetylethylenediamine, which is a stable water soluble compound. Thus, two moles of peracetic acid are generated for every mole of tetraacetylethylenediamine. Peracetic acid is a fast acting bleaching agent even at low wash temperatures.

(CH₃C(O))₂NCH₂CH₂N(C(O)CH₃)₂+H₂O₂→(CH₃C(O))₂NCH₂CH₂NH(C(O)CH₃)+CH₃CO₃H (1)

(CH₃C(O))₂NCH₂CH₂NH(C(O)CH₃)+H₂O₂→(CH₃C(O))HNCH₂CH₂NH(C(O)CH₃)+CH₃CO₃H (2)

The rate of peracetic acid generation is determined by the pH and temperature of the application environment, the molar ratio of hydrogen peroxide to bleach activator and nature of the bleach activator. The rate increases with pH, temperature and molar excess of hydrogen peroxide. Thus, its generation can be tailored to the needs of a given application through appropriate formulation.

In recent years significant changes have been realised in laundry detergent technology, driven by the leading manufacturers, with a shift from powdered to liquid products in all major geographic markets as well as a shift to lower washing temperatures. However, these liquid products do not include a bleaching system, and therefore their efficacy with respect to stain removal is compromised since the common bleach activators essential for low temperature stain removal are unstable in these media.

The degradation of tetraacetylethylenediamine in aqueous media may be described by a characteristic half-life, which is temperature and pH dependent. The half-life decreases with both increasing temperature and increasing pH. At 37° C., a common temperature employed for the accelerated ageing of the liquid media, the half-life has been determined as 6½ days at pH 5.7 but only 6½ seconds at pH 11.3 in an aqueous medium free of detergent components. However, the liquids are well matched to the consumers' expectations of lower temperatures and reduced wash cycle times, where unacceptable visible detergent residues, which are particularly obvious on dark colours, may be encountered with powdered products.

A multitude of chemical and physical parameters may be applied to the detailed description of any given liquid detergent product. However, when considering the plethora of products available to the consumer, a description of their pH, microstructure and water content usually suffices. Thus, products may be acidic, neutral or alkaline, be unstructured or structured (to give a gel) and either include no water, i.e. zero water (anhydrous), or have a low (5-15%), medium (30-35%) or high (60-70%) water content. All product types are encountered in the market. However, the preferred product or products will be determined based on the consumer habits of a particular geographic market and the expected function of the product.

The coating and encapsulation of detergent components with various inorganic and organic materials has been widely documented in the art. By way of example, WO 94/15010 (The Proctor & Gamble Company) discloses a solid peroxyacid bleach precursor composition in which particles of peroxyacid bleach precursor are coated with a water-soluble acid polymer, defined on the basis that a 1% solution of the polymer has a pH of less than 7.

Likewise, WO 94/03568 (The Proctor & Gamble Company) discloses a granular laundry detergent composition having a bulk density of at least 650 g/l, which comprises discrete particles comprising from 25-60% by weight of anionic surfactant, inorganic perhydrate bleach and a peroxyacid bleach precursor, wherein the peroxyacid bleach precursor is coated with a water soluble acidic polymer.

U.S. Pat. No. 6,225,276 (Henkel Kommanditgesellschaft auf Aktien) discloses a solid particulate detergent composition comprising a coated bleaching agent that dissolves in water irrespective of pH, a bleach activator coated with a polymeric acid that only dissolves at pH values above 8, and an acidifying agent.

U.S. Pat. No. 5,972,506 (BASF Aktiengesellschaft) discloses microcapsules containing bleaching agents. The microcapsules are obtained by polymerizing a mixture of monomers in the oil phase of a stable oil-in-water emulsion in the presence of free radical polymerization initiators.

WO 97/14780 (Unilever NV) discloses an encapsulated bleach particle comprising a coating including a gelled polymer material, and a core material which is selected from a peroxygen bleach compound, a bleach catalyst and a bleach precursor. The gelled polymer has a molecular structure that is partially or fully cross-linked, such as for example, agar, alginate, carrageenan, casein, gellan gum, gelatine, pectin, whey proteins, egg protein gels and the like.

WO 98/16621 (Warwick International Group Ltd) discloses a process for encapsulating a solid detergent component from an oil-in-water emulsion by forming a polymer film at the oil/water interface by condensation polymerisation. Suitable polymer films include polyamide, polyester, polysulphonamide, polyurea and polyurethane.

WO 98/00515 (The Proctor & Gamble Company) discloses non-aqueous, particulate-containing liquid laundry cleaning compositions which are in the form of a suspension of particulate material comprising peroxygen bleaching agents and coated peroxygen bleach activators. The coating material is soluble in water, but insoluble in non-aqueous liquids, and is selected from water soluble citrates, sulfates, carbonates, silicates, halides and chromates.

WO 93/24604 (BP Chemicals Ltd) discloses an encapsulated active substrate comprising a bleach and/or a bleach activator releasably encapsulated in a coating of an alkali metal carbonate or bicarbonate and an outer encapsulating coating of a metal salt of an inorganic salt.

U.S. Pat. No. 6,107,266 (Clariant GmbH) discloses a process for producing coated bleach activating granules in which bleach activator base granules are coated with a coating substrate and are simultaneously and/or subsequently thermally conditioned. The coating substance is selected from C₈-C₃₁ fatty acids, C₈-C₃₁ fatty alcohols, polyalkylene glycols, non-ionic surfactants and anionic surfactants.

However, despite the breadth of the above described technologies, a bleaching system ideally suited for inclusion in liquid laundry products has not yet been developed. One practical solution is the formulation of a two component liquid product, which distributes the incompatible ingredients between them (to give two stable components), which are mixed on dispensing. Such systems have already been introduced to the market. However, their packaging is substantially more expensive than a standard single chamber unit, which combined with the poor consumer feedback, has led to the conclusion that a fully formulated product must be offered to satisfy the market.

The present invention seeks to provide a composite material in which a solid bleach activator is physically isolated, for example, from the bulk of other laundry product components, by virtue of encapsulation in a polymeric coating.

### STATEMENT OF INVENTION

A first aspect of the invention relates to a composite comprising:

(i) one or more core units comprising a bleach activating agent; and

(ii) an alkali soluble polymer coating on the surface of said one or more core units.

Advantageously, the composite of the invention is suitable for inclusion in acidic or neutral liquid laundry products as a coated suspension, but is readily soluble in the alkaline wash environment, whereupon the bleach activator will be released and act in the usual manner in combination with active oxygen bleaching agents and/or hydrogen peroxide.

A second aspect of the invention relates to a process for preparing a composite as described above, said process comprising applying the alkali soluble polymer coating to the surface of said one or more core units.

A third aspect of the invention relates to a laundry product comprising a composite as described above.

A fourth aspect of the invention relates to a method of preparing a laundry product as described above, said method comprising admixing a composite according to the invention with one or more conventional laundry product components.

A fifth aspect of the invention relates to the use of a composite as described above as an additive in a laundry product.

A sixth aspect of the invention relates to a method of generating peracetic acid in situ, said method comprising subjecting a composite as described above to a pH value at or above the pKₐ value of the alkali soluble polymer coating, in the presence of a bleaching agent.

A seventh aspect of the invention relates to a bleaching system comprising a composite according to the invention and a bleaching agent.

An eighth aspect of the invention relates to an alkali soluble polymer suitable for coating a bleach activating agent, wherein said alkali soluble polymer is as defined above.

### DETAILED DESCRIPTION

Polymeric Coating Material

As mentioned above, one aspect of the invention relates to a composite comprising:

(i) one or more core units comprising a bleach activating agent; and

(ii) an alkali soluble polymer coating on the surface of said one or more core units.

The alkali soluble polymers for use in the coating are preferably insoluble at acidic and neutral pH values (e.g. preferably below their pKₐ value) and soluble at basic pH values (e.g. preferably at or above their pKₐ value).

In one preferred embodiment the composite comprises a plurality of core units comprising a bleach activating agent, said core units are coated with an alkali soluble polymer.

Advantageously, the composite of the invention comprises a coating of either a single alkali soluble polymer (preferably a copolymer, more preferably an acrylic copolymer) or mixtures of such polymers to give a coated product, whose stability is sufficient to permit its incorporation into acidic or neutral liquid detergent media intended for domestic, commercial and institutional use, where the media may be unstructured or structured and include either no water or some water. The polymer coating is insoluble in the product environment and presents an effective barrier to the components of the medium including anionic, nonionic and cationic surfactants, active oxygen bleaching agents, hydrogen peroxide, water and any other additives, but is soluble in the alkaline wash environment, whereupon the bleach activator will be released and act in the usual manner in combination with the active oxygen bleaching agent and/or hydrogen peroxide.

Preferably, the alkali soluble polymer is insoluble at pH values below its pKₐ value, and soluble at pH values at or above its pKₐ value.

A further aspect of the invention relates to a method of generating peracetic acid in situ, said method comprising subjecting a composite as described above to a pH value at or above the pKₐ value of the alkali soluble polymer coating in the presence of a bleaching agent.

The composites according to the present invention typically contain from about 10% to about 75%, preferably from about 15% to about 50% and more preferably from about 25% to about 40% of said alkali soluble polymer coating by weight of the total composite.

Preferably, the coating is present in a thickness of from about 5 μM to about 90 μM, preferably about 8 μM to about 40 μM and most preferably from 15 μM to 30 μM.

In a highly preferred embodiment, at least a portion of the core units are completely encapsulated by the alkali soluble polymeric coating. More preferably, substantially all, or all, of the core units are completely encapsulated by the alkali soluble polymeric coating. However, the invention also encompasses composites in which at least a portion of the core units are only partially coated, for example, composites in which at least a proportion of the core units are partially coated to a sufficient degree to still exhibit the desired functional characteristics of the invention, namely, so that the coating presents an effective barrier to the remaining components of the medium, but is soluble in the alkaline wash environment, whereupon the bleach activator will be released.

In one preferred embodiment of the invention, the alkali soluble polymer is prepared from monomers having only one polymerisable double bond. Suitable monomers having only one polymerisable double bond include, but are not limited to, styrene and substituted styrenes such as α-methyl styrene, methyl styrene, t-butyl styrene, alkyl esters of mono-olefinically unsaturated dicarboxylic acids such as di-n-butyl maleate and di-n-butyl fumarate; vinyl esters of carboxylic acids such as vinyl acetate, vinyl propionate, vinyl laurate and vinyl esters of versatic acid such as VeoVa 9 and VeoVa 10 (VeoVa is a trademark of Shell); acrylamides such as methyl acrylamide and ethyl acrylamide; methacrylamides such as methyl methacrylamide and ethyl methacrylamide; nitrile monomers such as acrylonitrile and methacrylonitrile; and esters of acrylic and methacrylic acid, preferably optionally substituted C₁₋₂₀alkyl and C₁₋₂₀cycloalky esters of acrylic and methacrylic acid, such as methyl acrylate, ethyl acrylate, n-butyl acrylate, 2-ethylhexyl acrylate, i-propyl acrylate, and n-propyl acrylate, methyl methacrylate, ethyl methacrylate, n-butyl methacrylate, 2-ethylhexyl methacrylate, i-propyl methacrylate, n-propyl acrylate, hydroxyethyl acrylate, hydroxyethyl methacrylate, N,N-dimethylaminoethyl acrylate and N,N-dimethylaminoethyl methacrylate.

In one preferred embodiment, the alkali soluble polymer is an acrylic copolymer.

Preferably, the acrylic copolymer is formed from monomers selected from, but not limited to, methylmethacrylate (MMA), ethylmethacrylate (EMA), butylmethacrylate (BMA), isobutylmethacrylate (iBMA), 2-ethylhexyl methacrylate (EHMA), isobornylmethacrylate (iBoMA), methylacrylate (MA), ethylacrylate (EA), butylacrylate (BA), 2-ethylhexylacrylate (EHA), styrene (STY), acrylic acid (AA), methacrylic acid (MAA) and sodium acrylate (SAA).

Preferably, the acrylic copolymer has a molecular weight of from about 20,000 Daltons to 500,000 Daltons, more preferably from about 40,000 Daltons to about 250,000 Daltons.

Preferably, the acrylic copolymer possesses a pKₐ value of from 3.0 to 10.0, more preferably from about 4.5 to about 9.5, and most preferably 1 unit greater than the pH of the detergent medium into which the coated composite is compounded and 1 unit less than the pH of the washing liquor.

Preferably, the acrylic copolymer has a glass transition temperature of from about −40° C. to about 100° C., more preferably from about 10° C. to about 80° C.

Preferably, the acrylic copolymer demonstrates a minimum film forming temperature of from about 0° C. to about 100° C., more preferably from about 10° C. to about 80° C.

In one preferred embodiment, the copolymer is a random copolymer.

In another preferred embodiment, the copolymer is a block copolymer.

Preferably, the copolymer is prepared from a mixture of at least one dissociating monomer and at least one non-dissociating monomer.

In one particularly preferred embodiment, the polymer is of general formula I,

R¹—[(X)ₓ—(Y)_{y}—(Z)_{z}]ₙ—R² (I)

wherein:

R¹ and R² are each independently bleach stable polymer end groups;

—(X)ₓ—(Y)_{y}—(Z)_{z}— is a polymer backbone formed from the polymerization of X′, Y′ and Z′;

X′ is a first non-dissociating monomer of formula R³R⁴C═CR⁵—CO—OR⁶ or R³R⁴C═CR⁵R⁶;

Y′ is a second non-dissociating monomer of formula R³R⁴C═CR⁵—CO—OR⁶ or R³R⁴C═CR⁵R⁶;

Z′ is a dissociating monomer of formula R³R⁴C═CR⁵—CO—OH or formula R³R⁴C═CR⁵—CO—O—(CH₂)ₙ—CO₂H;

R³, R⁴ and R⁵ are each independently hydrogen or an inert aliphatic or aromatic organic moiety;

R⁶ is an inert aliphatic or aromatic organic moiety;

x is an integer from 30 to 90;

y is an integer from 0 to 50;

z is an integer from 10 to 60;

wherein the sum of x+y+z=100; and

n is an integer from 2 to 60.

Suitable polymer end groups include hydrogen, linear and branched alkyl groups, preferably C₁₋₅₀-alkyl, more preferably C₁₋₂₀-alkyl or C₁₋₁₀-alkyl, and moieties derived from the free radical polymerisation initiators employed in the preparation of the polymer, including sulphonate and azo groups.

Suitable inert aliphatic or aromatic organic moieties include unsubstituted or substituted C₁₋₅₀-alkyl or C₆₋₁₀-aryl, more preferably C₁₋₂₀-alkyl, C₁₋₁₀-alkyl or C₆₋₈-aryl. Preferably, the moieties may be substituted with a C₁₋₁₀ linear or branched alkyl group, preferably a C₁₋₆ linear or branched alkyl group, or a C₆₋₁₀-aryl group.

In one particularly preferred embodiment, X′ and/or Y′ are R³R⁴C═CR⁵R⁶, wherein R³, R⁴ and R⁵ are H and R⁶ is unsubstituted C₆-aryl or a C₆-aryl substituted with a C₁₋₆ linear or branched alkyl-group. Preferably, only one of X′ or Y′ has the formula R³R⁴C═CR⁵R⁶, i.e. the other is R³R⁴C═CR⁵—CO—OR⁶.

As used herein, the term “dissociating monomer” refers to a monomer that gives rise to polymer chains characterised by the presence of a carboxylic acid residue (—CO₂H). The following acid-base dissociation may be described:

—CO₂H⇄—CO₂⁻+H⁺

This equilibrium is characterised by its pKₐ value.

Under acidic conditions (e.g. where pH<pKₐ), an uncharged carboxylic acid (—CO₂H) residue is encountered, which will render an uncharged and insoluble polymer chain, essential to the protection of the bleach activator core of the composite within the detergent medium. However, under alkaline conditions (e.g. where pH>pKₐ), a charged anionic carboxylate group (—CO₂⁻) will be encountered, which gives rise to a charged and readily water soluble polymer backbone, essential for the release of the bleach activator in the wash liquor.

Examples of useful dissociating monomers include, but are not limited to, acrylic acid (AA), methacrylic acid (MAA) and β-carboxyethyl-acrylate (BCEA).

As used herein, the term “non-dissociating monomer” refers to a monomer that contains a —CO—OR⁶ group, where R⁶ is other than hydrogen (e.g. where R⁶ is an inert aliphatic or aromatic organic moiety) or a monomer of formula R³R⁴C═CR⁵R⁶, thus the monomer is not capable of acid-base dissociation. The chemical/physical characteristics of the non-dissociating monomer segments of the polymer chains are independent of pH. They are typified by monomers such as EA, BA, BMA, EHA, STY, MMA and the like. Preferably, the copolymer backbone is formed from more than one non-dissociating co-monomer to ensure an adequate balance of physical (minimum film forming and glass transition temperatures) and barrier properties.

As mentioned above, preferably the polymer is a copolymer of at least one dissociating monomer and at least one non-dissociating monomer.

Preferably, the bulk of the polymer (e.g. up to 95%) comprises non-dissociating monomers (such as BMA, EHA, STY, MMA) with the balance (e.g. up to 20%) comprising dissociating monomers (such as AA, MAA, BCEA). The non-dissociating monomers provide the essential chemical resistance and barrier characteristics, whereas the dissociating monomers give the necessary pH switch to insolubility/solubility.

In one preferred embodiment, the copolymer comprises from about 70 to about 99 weight % of non-dissociating monomers (such as BMA, EHA, STY, MMA), preferably from about 80 to about 99 weight, more preferably from about 80 to about 95 weight %.

Preferably, the copolymer comprises from about 1 to about 30 weight % of dissociating monomers (such as AA, MAA, BCEA), preferably from about 1 to about 20 weight %, more preferably from about 5 to about 20 weight %.

Advantageously, the alkali soluble polymers of the invention maximise the barrier properties of the polymer coating towards the amphiphilic species found in liquid detergent media. This is achieved by controlling the characteristics of the coating through the selection of the most appropriate non-dissociating monomers, whilst at the same time not impairing the ability to release the core material under alkaline conditions. Practically this is achieved by balancing the choice and proportion of hydrophilic and hydrophobic non-dissociating comonomers employed in the synthesis of the coating polymer.

Examples of useful hydrophilic non-dissociating comonomers are MMA, MA, EA, EMA.

Examples of useful hydrophobic non-dissociating comonomers are STY, EHA, BMA.

In another preferred embodiment, the alkali soluble polymer is an acrylic copolymer formed from monomers selected from, but not limited to, methylmethacrylate (MMA), styrene (STY), ethylmethacrylate (EMA), butylmethacrylate (BMA), isobutylmethacrylate (iBMA), methyl acrylate (MA), butylacrylate (BA), 2-ethylhexylacrylate (EHA), acrylic acid (AA), methacrylic acid (MAA) and β-carboxyethylacrylate (BCEA).

In one highly preferred embodiment, the acrylic copolymer is formed from a mixture of monomers selected from butylmethacrylate (BMA), 2-ethylhexylacrylate (EHA), methacrylic acid (MAA).

In another highly preferred embodiment, the acrylic copolymer is formed from a mixture of monomers selected from ethylmethacrylate (EMA), 2-ethylhexylacrylate (EHA), methacrylic acid (MAA).

In another highly preferred embodiment, the acrylic copolymer is formed from a mixture of monomers selected from styrene (STY), 2-ethylhexylacrylate (EHA), methacrylic acid (MAA).

In another highly preferred embodiment, the acrylic copolymer is formed from a mixture of monomers selected from methylmethacrylate (MMA), butylmethacrylate (BMA) or butylacrylate (BA), acrylic acid (AA).

In one especially preferred embodiment, the acrylic copolymer is formed from a mixture of monomers selected from butylmethacrylate (BMA), 2-ethylhexylacrylate (EHA) and methacrylic acid (MAA) in a ratio of 70:10:20 (BMA:EHA:MAA).

In one preferred embodiment the acrylic copolymer is formed from a mixture of from about 45 to about 90 weight % EMA, from about 10 to about 50 weight % EHA or BA and from about 5 to about 15 weight % MAA, preferably from about 50 to about 85 weight % EMA, from about 10 to about 45 weight % EHA or BA and from about 5 to about 12 weight % MAA, more preferably from about 55 to about 80 weight % EMA, from about 10 to about 35 weight % EHA or BA and from about 6 to about 12 weight % MAA.

In one preferred embodiment the acrylic copolymer is formed from a mixture of from about 55 to about 95 weight % BMA, from about 1 to about 20 weight % EHA and from about 5 to about 15 weight % MAA, preferably from about 60 to about 90 weight % BMA, from about 1 to about 15 weight % EHA and from about 5 to about 15 weight % MAA, more preferably from about 70 to about 85 weight % BMA, from about 5 to about 13 weight % EHA and from about 5 to about 12 weight % MAA.

In one preferred embodiment the acrylic copolymer is formed from a mixture of from about 20 to about 80 weight % STY, from about 20 to about 60 weight % EHA and from about 1 to about 35 weight % MAA, preferably, 25 to about 65 weight % STY, from about 25 to about 50 weight % EHA and from about 5 to about 25 weight % MAA.

In one preferred embodiment the acrylic copolymer is formed from a mixture of from about 60 to about 80 weight % MMA, from about 10 to about 30 weight % BA and from about 1 to about 15 weight % AA, preferably, 65 to about 75 weight % MMA, from about 15 to about 35 weight % BA and from about 5 to about 10 weight % AA.

In one preferred embodiment the acrylic copolymer is formed from a mixture of from about 60 to about 80 weight % BMA, from about 10 to about 30 weight % MMA and from about 1 to about 15 weight % AA, preferably, 65 to about 75 weight % BMA, from about 15 to about 35 weight % MMA and from about 5 to about 10 weight % AA.

In one preferred embodiment of the invention, the alkali soluble polymer coating comprises a mixture of two or more acrylic copolymers as described herein.

The alkali soluble polymers of the invention are conveniently produced from a wide range of starting monomers by a number of synthetic routes including bulk, solution, suspension and emulsion polymerisation. The polymers are most conveniently produced by emulsion polymerisation.

The choice and quantity of the monomers employed will determine the characteristics of the polymer; hydrophilic/hydrophobic balance, softness/hardness, glass transition temperature (T_{g}) and solution characteristics. Particularly preferred monomers are selected from, but not limited to, methylmethacrylate (MMA), ethylmethacrylate (EMA), butylmethacrylate (BMA), isobutylmethacrylate (iBMA), 2-ethylhexyl methacrylate (EHMA), isobornylmethacrylate (iBoMA), methylacrylate (MA), ethylacrylate (EA), butylacrylate (BA), 2-ethylhexylacrylate (EHA), styrene (STY), acrylic acid (AA), methacrylic acid (MAA) and sodium acrylate (SAA), where, for example, acrylic acid (AA) would be considered a hydrophilic monomer, whereas 2-ethylhexylacrylate would be considered to be a hydrophobic monomer, and where butylacrylate (BA) would be a soft monomer, but styrene (STY) a hard monomer. When selecting the monomers for the execution of a polymer synthesis, the reactivity ratio of the monomer combinations must also be taken into account to ensure that the desired distribution of monomers is achieved whether that is a blocky or random distribution.

Such polymers may be tailored to give a desirable balance of properties including controlled solubility as a function of pH, with insolubility observed at acidic and neutral pH values (below their pKₐ values) and solubility at basic pH values (above their pKₐ values). Ideally, the polymers give tough, non-tacky, flexible dry films that demonstrate good adhesion to the core material and facilitate the preparation of a free flowing coated product robust to brittle fracture and coating failure, whilst demonstrating low water uptake from acidic aqueous media and good barrier properties.

Such materials are commercially available from various polymer suppliers including, for example, DSM NeoResins (Waalwijk, The Netherlands). The behaviour of several commercially available alkali soluble acrylic copolymers and mixtures thereof, has been explored; for example, NeoCryl BT-26 (T_{g}=34° C.), NeoCryl BT-27 (T_{g}=16° C.) and NeoCryl BT-36 (T_{g}=61° C.). However, the choice of commercially available polymers is limited to copolymer compositions tailored to applications significantly different to that of encapsulating bleach activators. Thus, a range of alkali soluble acrylic copolymers were prepared and evaluated by the inventors.

Emulsion polymerisation may be conducted at temperatures from about 20° C. to about 95° C. Preferably, emulsion polymerisation may be conducted at a temperature of at least about 70° C., more preferably from about 75° C. to about 85° C.

Preferably, the monomers are selected from methylmethacrylate (MMA), ethylmethacrylate (EMA), butylmethacrylate (BMA), isobutylmethacrylate (iBMA), methyl acrylate (MA), butylacrylate (BA), 2-ethylhexylacrylate (EHA), styrene (STY), acrylic acid (AA), methacrylic acid (MAA) and β-carboxyethylacrylate (BCEA). The system is preferably stabilised with anionic surfactants including, but not limited to, sodium lauryl sulphate (SLS), sodium benzene alkyl sulphonate (SBAS) and sodium dioctylsulfosuccinate (SDSS). The polymerisation is preferably initiated using a free radical initiator. Suitable initiators include, but are not limited to, persulphates, percarbonates, inorganic peroxides, organic peroxides (such as dialkyl peroxides, acyl peroxides, alkyl hydroperoxides, peroxy esters), hydroperoxides, azo compounds and cobalt complexes. More preferred initiators include potassium persulphate, ammonium persulphate, sodium persulphate, hydrogen peroxide, benzoyl peroxide, cumene hydroperoxide, t-butyl hydroperoxide, di-t-butyl peroxide, t-butyl perbenzoate, azoisobutyronitrile, cobalt II and cobalt II complexes of porphyrins, dioximes and benzildioxime diboron compounds. Other suitable initiators include azo-iso-butyronitrile, dimethyl 2,2′-azo bis-isobutylate, hydrogen peroxide and benzoyl peroxide.

Chain transfer agents (CTA) are typically employed to control molecular weight. Suitable chain transfer agents include, but are not limited to, mercaptans, for example, methyl-3-mercapto propionate (MMP), lauryl mercaptan (LM) or primary octyl mercaptan (POM).

The resulting latices demonstrated an average hydrodynamic diameter (by photon correlation spectroscopy) of 80-475 nanometers, whilst the isolated polymers demonstrated glass transition temperatures (T_{g}) in the range 8-85° C. Further details of polymer synthesis may be found in the accompanying examples section.

Bleach Activator

The present invention relates to a composite comprising a bleach activator (also referred to as a peroxyacid bleach precursor). Preferably, the bleach activator is a solid bleach activator.

The composites according to the present invention typically contain from about 25% to about 90%, preferably from about 50% to about 85% and most preferably from about 60% to about 75% of said bleach activator by weight of the total composite.

The bleach activators employed in the invention are capable of reacting with a peroxygen compound in aqueous solution to form in situ a peroxyacid corresponding to the bleach activator structure.

Typically, the bleach activators of the present invention comprise precursors containing one or more N-acyl or O-acyl groups, which can be selected from a wide range of classes. Suitable preferred classes include anhydrides, esters, imides and acylated derivatives of imidazoles and oximes, acylated triazine derivatives, acylated glycol urils, N-acyl-imides, acylated phenol sulfonates, carboxylic anhydrides, acylated polyhydric alcohols, acylated sugar derivatives, acetylated glycamine, gluconolactone and N-acylated lactams. Examples of useful materials within these classes are known in the art. The most preferred classes are esters, such as those disclosed in GB 836988, GB 864798, GB 907356, GB 907358, GB 1246339, GB 1147871 and GB 2143231, and imides such as those disclosed in GB 855735 and GB 1246338. These are discussed in more detail below:

a. Esters of phenols and substituted phenols, for example, as described in GB 836988. An example is phenylacetate.

b. Esters of monohydric aliphatic alcohols, for example, as described in GB 836988. An example is trichloroethylacetate.

c. Esters of polyhydric aliphatic alcohols, for example, as described in GB 836988. An example is mannitol hexaacetate.

d. Esters of mono- and disaccharides, for example, as described in GB 836988. An example is fructose pentaacetate.

e. Esters containing 2 ester groups, for example, as described in GB 836988. An example is benzaldehyde diacetate.

f. Esters of monobasic carboxylic acids, for example, as described in GB 864798. An example is sodium p-acetoxybenzene sulphonate.

g. N-diacylated amines, for example, as described in GB 907356 and GB 907358. An example is diacetylethylamine.

h. N-diacylated ammonias, for example, as described in GB 907356 and GB 907358. An example is diacetamide.

i. N-diacylated amides, for example, as described in GB 907356, GB 907358 and GB 855735. Examples include N-formyldiacetamide and N,N-diacetylaniline.

j. N-diacylated urethanes, for example, as described in GB 907356 and GB 907358. An example is N,N-diacetylethylurethane.

k. N-diacylated hydrazines, for example, as described in GB 907356 and GB 907358. An example is triacetylhydrazine.

l. N-triacylated alkylene diamines, for example, as described in GB 907356 and GB 907358. An example is N¹,N¹,N²-triacetylmethylenediamine.

m. N-tetraacylated alkylene diamines, for example, as described in GB 907356. Examples include N¹,N¹,N²,N²-tetraacetylmethylenediamine and N¹,N¹,N²,N²-tetraacetylethylenediamine (TAED).

n. N-diacyl derivatives of semicarbazide, thiosemicarbazide and dicyanodiamide, for example, as described in GB 907356 and GB 907358.

o. Tetraacylated glycol-urils, for example, as described in GB 124338 and GB 1246339. Examples include 1,3,4,6-tetraacetyl glycol-uril and 1,3,4,6-tetrapropionyl glycol-uril.

p. Acyl alkyl and acyl benzene sulphonates, for example, as described in GB 1147871. An example is sodium 2-acetoxy-5-hexyl-benzene sulphonate.

q. Acyloxybenzene sulphonates, for example, as described in GB 2143231. Examples include sodium 3,5,5-trimethyl hexanoyloxybenzene sulphonate, sodium 2-ethyl hexanoyloxybenzene sulphonate and sodium nonanoyloxybenzene sulphonate (SNOBS).

Preferred examples also include ethylene glycol diacetate, 2,4-diacetoxy-2,5-dihydrofuran, acetylated sorbitol, acetylated mannitol and mixtures thereof.

Particularly preferred precursor compounds are the N,N,N′N′-tetra acetylated compounds of formula (CH₃CO)₂—N—(CH₂)ᵣ—N—(COCH₃)₂, wherein r is zero or an integer from 1 to 6. Examples include tetraacetylmethylenediamine (TAMD) wherein r is 1, tetraacetylethylenediamine (TAED) wherein r is 2, and tetraacetylhexylenediamine (TAHD) wherein r is 6. These and analogous compounds are described in GB-907356. The most preferred bleach activator is TAED.

Solid bleach activators useful in the present invention typically have a melting point of >30° C. and preferably >40° C.

In one preferred embodiment the bleach activator may be in particulate form. Preferably, the bleach activator particles have a maximum dimension in the range of from about 25 microns to about 1500 microns, more preferably from about 50 microns to about 1000 microns, and more preferably still from about 150 microns to about 600 microns.

In one preferred embodiment the bleach activating agent may be in granulate form.

In one highly preferred embodiment of the invention, the bleach activating agent is combined with one or more granulating or binding agents. Suitable granulating or binding agents will be familiar to the skilled artisan and include, for example, alkali soluble polymers (as described for the coating) and copolymers synthesised with other dissociating monomers, which demonstrate lower pKₐ values than the carboxylic acid moiety, of acrylic acid (AA), methacrylic acid (MAA) and β-carboxyethylacrylate (BCEA). Especially preferred monomers include, but are not limited to, 2-aminopropylmethyl sulphonic acid (AMPS) and sodium styrene sulphonate (NaSS).

Preferably, the bleach activating agent is combined with a granulating polymer. Preferably, the granulate comprises between about 75% to 95% by weight of bleach activating agent and from about 1 to about 25% by weight of said granulating polymer, more preferably, between about 85% to 95% by weight of bleach activating agent and from about 1 to about 15% by weight of said granulating polymer, most preferably, about 90% of bleach activating agent and from about 2 to about 10% of said granulating polymer.

The granulating polymer is preferably selected from polyacrylic acid, polyvinyl alcohol, an alkali soluble polymer as described in the present invention, an alkali soluble polymer possessing a pKa value equal to or less than that of the coating material, and combinations thereof.

Thus, in one preferred embodiment, the core units comprise granulated bleach activating agent, a granulating agent selected from polyacrylic acid, polyvinyl alcohol and an alkali soluble polymer as described above.

As mentioned above, in one highly preferred embodiment of the invention, the bleach activator is tetraacetylethylenediamine. Tetraacetylethylenediamine, commonly abbreviated to TAED, is an organic compound having the formula (CH₃C(O))₂NCH₂CH₂N(C(O)CH₃)₂. It is a colourless crystalline solid demonstrating slight solubility in water and a melting point of 149-154° C. TAED is susceptible to decomposition in humid and aqueous environments and must be maintained in a dry environment during transport, storage and formulation with other materials. Although the invention primarily focuses on composites comprising tetraacetylethylenediamine, the skilled person will appreciate that the teachings disclosed herein apply equally to other bleach activators.

Scanning electron microscopy permits visualisation of the individual TAED particles. Two different forms have been identified:
(i) Tetraacetylethylenediamine in the form of a mixture of irregularly shaped particles; whose maximum dimensions fall within the range of from about 5 microns to about 250 microns; and
(ii) Granulated tetraacetylethylenediamine found as aggregates of irregularly shaped particles with most aggregates having a maximum dimension of greater than about 200 microns. The surface of the granulate is found to be uneven and porous.

Thus, in one particularly preferred embodiment of the invention, the tetraacetylethylenediamine is in particulate form. For this embodiment, typically the particles are irregularly shaped with maximum dimensions preferably from about 5 to about 250 microns.

In another particularly preferred embodiment of the invention, the tetraacetylethylenediamine is granulated. Preferably, the granulated tetraacetylethylenediamine is in the form of aggregates of irregularly shaped particles with having a maximum dimension of greater than about 200 microns.

In one highly preferred embodiment of the invention, the tetraacetylethylenediamine is combined with one or more granulating or binding agents. Suitable granulating or binding agents will be familiar to the skilled artisan and include, for example, alkali soluble polymers (as described for the coating) and copolymers synthesised with other dissociating monomers, which demonstrate lower pKₐ values than the carboxylic acid moiety, of acrylic acid (AA), methacrylic acid (MAA) and β-carboxyethylacrylate (BCEA). Especially preferred monomers include, but are not limited to, 2-aminopropylmethyl sulphonic acid (AMPS) and sodium styrene sulphonate (NaSS).

In one highly preferred embodiment, the granulating agent is polyacrylic acid or polyvinyl alcohol, or combinations thereof. Alternatively, or in addition to, the core may also comprise an alkali soluble polymer corresponding to the coating material described above or an alkali soluble polymer possessing a pKa value equal to or less than that of the coating material.

Thus, in one preferred embodiment, the core units comprise granulated tetraacetylethylenediamine, a granulating agent selected from polyacrylic acid, polyvinyl alcohol and an alkali soluble polymer as described above.

The particle size distribution of the core material has an important influence on the results of the coating process. Optimum results are obtained by balancing the need for the economic use of the coating materials and ease of processing, which precludes the use of the smallest particle sizes (with their associated large total surface per unit mass), and suspension of the particle in the final product, which precludes the use of the largest particle sizes. Analysis of the competing factors suggests that a good balance is achieved with particles having a maximum dimension in the range 50 microns to 500 microns. Particles of the desired size may be simply obtained by classifying or granulating tetraacetylethylenediamine.

Coating Process

A further aspect of the invention relates to a process for preparing a composite as defined above, said process comprising applying the alkali soluble polymer coating to the surface of said one or more core units.

In one preferred embodiment, the core units are prepared by co-agglomerating a granulating or binding agent with the bleach activator prior to coating the core units with the alkali soluble polymer.

Encapsulation may be carried out by any suitable means and the method is not critical to the invention.

For example, the coating material may be sprayed on as a molten material or as a solution, latex or dispersion in a solvent/carrier liquid which is subsequently removed by evaporation. The coating material can also be applied as a powder coating e.g. by electrostatic techniques, although this is less preferred as the adherence of powdered coating material is more difficult to achieve and can be more expensive.

Molten coating is a preferred technique for coating materials of melting point <80° C. but is less convenient for higher melting points (i. e. >100° C.). For coating materials of melting point >80° C., spraying on as a solution, latex or dispersion are preferred. Organic solvents such as ethyl and isopropyl alcohol can be used to form the solutions or dispersions, although this will necessitate a solvent recovery stage in order to make their use economic. However, the use of organic solvents also gives rise to safety problems such as flammability and operator safety and thus aqueous solutions, latex or dispersions are preferred.

Aqueous solutions are particularly advantageous as the coating materials herein have a high aqueous solubility, provided the solution has a sufficiently low viscosity to enable it to be handled. Preferably a concentration of from about 5% to about 50% and preferably from about 10% to about 25% by weight of the coating material in the solvent is used in order to reduce the drying/evaporation load after surface treatment has taken place. The treatment apparatus can be any of those normally used for this purpose, such as inclined rotary pans, rotary drums and fluidised beds.

In one highly preferred embodiment, the coating is applied to the cores either by fluid bed coating or fluid bed drying. The polymer is preferably applied to the core units as an alkaline coating solution or as an acidic latex. In the embodiment where the polymer is applied as an alkaline coating solution, preferably the solution further comprises a stabilizer, for example, ammonia. Aqueous alkaline solutions of the polymer are prepared by neutralisation of the acidic latex. Neutralisation with volatile amines, such as ammonia, trimethyl amine, triethyl amine, ethanolamine and dimethylethanolamine, is preferred as the volatile component is readily lost and a robust polymer coating is readily achieved. Typically neutralisation is accompanied by clarification of the coating mixture, from an opaque latex to a clear or hazy solution, and an increase in viscosity. Additional solvent may be added to reduce the polymer concentration and solution viscosity and so obtain a solution suitable for further processing.

In fluid bed coating the particulate core material is fluidised in a flow of hot air and the coating solution or latex sprayed onto the particles and dried, where the coating solution or latex may be applied by top spray coating, bottom spray (Wurster) coating or tangential spray coating, where bottom spray (Wurster) coating is particularly effective in achieving a complete encapsulation of the core. In general, a small spray droplet size and a low viscosity spray medium promote uniform distribution of the coating over the particles.

In fluid bed drying the particulate core material is mixed with the coating solution or latex and the resulting moist product introduced to the fluid bed dryer, where it is held in suspension in a flow of hot air, where it is dried. Such systems are available from several suppliers including GEA Process Engineering (Bochum, Germany) and Glatt Process Technology (Binzen, Germany).

In one preferred embodiment, the process of the invention comprises the step of preparing the alkali soluble polymer by emulsion polymerisation.

Preferably, for this embodiment, the process comprises preparing the alkali soluble acrylic copolymer by emulsion polymerisation from a reaction mixture comprising monomers selected from, but not limited to, methylmethacrylate (MMA), ethylmethacrylate (EMA), butylmethacrylate (BMA), isobutylmethacrylate (iBMA), methyl acrylate (MA), butylacrylate (BA), 2-ethylhexylacrylate (EHA), styrene (STY), acrylic acid (AA), methacrylic acid (MAA) and β-carboxyethylacrylate (BCEA).

In one preferred embodiment, the reaction mixture is stabilised with an anionic surfactant selected from sodium lauryl sulphate (SLS), sodium benzene alkyl sulphonate (SBAS) and sodium dioctylsulfosuccinate (SDSS).

In one preferred embodiment, the emulsion polymerisation is initiated with ammonium persulphate or tertiarybutylhydroperoxide.

In one preferred embodiment, the reaction mixture further comprises a chain transfer agent (CTA), preferably methyl-3-mercapto propionate (MMP).

When considering the characteristics of the resulting polymer coating, the following parameters are critical:
1. The mass of polymer coating applied per unit mass of the core.
2. The thickness of the polymer coating over the core.
3. The distribution of the polymer coating over the core.
4. The barrier created by the polymer coating to any species that may interact with and degrade the bleach activator.

The above parameters may be conveniently assessed through various qualitative and quantitative tests.

The results of the coating process are determined by the interaction of a combination of material and process parameters. In spray coating the following have been found to be important:
1. Composition and particle size distribution of the core.
2. The glass transition temperature of the polymer.
3. Delivery of the alkali soluble polymer as either a solution or a latex.
4. The solids content of the coating solution or latex.
5. The minimum film formation temperature of the latex or polymer solution.
6. The dosing rate of the coating solution or latex to the fluidised bed.
7. The delivery of the coating solution or latex by bottom spray or top spray.
8. The mass of polymer applied per unit mass of the core.
9. The inlet temperature of the air maintaining the fluidised bed.
10. The difference between the polymer's glass transition temperature and inlet air temperature of the air flow maintaining the fluidised bed.

Laundry Products

A further aspect of the invention relates to a laundry product comprising a composite as described above.

Composites in accordance with the present invention can be used in a variety of applications. Thus, the composites may themselves be incorporated into other solid compositions such as tablets, extrudates and agglomerates. The composites can also be suspended in aqueous and non-aqueous liquid compositions in which the alkali soluble polymer coating is insoluble and inert.

The preferred application for the composites of the invention is as components of liquid detergent compositions, particularly the so-called concentrated detergent compositions that are added to a washing machine by means of a dosing device placed in the machine drum with the soiled fabric load.

One preferred embodiment of the invention therefore relates to a liquid laundry product.

Preferably, the laundry product is an acidic or neutral liquid laundry product, more preferably, an acidic liquid laundry product.

In one preferred embodiment, the laundry product is a detergent composition, more preferably still, a liquid detergent composition. Typically, the liquid detergent composition will include water (from 0% to 70%) and bleach boosting additive products may contain up to 90% water.

In an alternative preferred embodiment, the laundry product is a powdered laundry product, more preferably, a powdered detergent composition.

Detergent compositions will typically contain from about 1% to about 15% of the composite of the invention, more preferably from about 2% to about 12% and even more preferably from about 4% to about 10% by weight of the total composition.

In one preferred embodiment, the laundry product further comprises one or more of an anionic surfactant, a non-ionic surfactant, a cationic surfactant, an active oxygen bleaching agent, hydrogen peroxide and water. Examples of suitable anionic, non-ionic surfactants are listed in U.S. Pat. No. 3,929,678 and WO 94/15010 whereas examples of suitable cationic surfactants are listed in U.S. Pat. No. 4,259,217 and WO 94/15010.

In one preferred embodiment, the laundry compositions of the invention further comprise particles of a peroxygen bleaching agent. Typically, the compositions comprise from about 1% to about 20%, more preferably from about 2% to about 10%, of the peroxygen bleaching agent by weight of the composition. The peroxygen bleaching agents are used in combination with the coated bleach activator and are typically inorganic compounds.

Suitable inorganic peroxygen compounds include alkali metal perborate and percarbonate materials, most preferably the percarbonates. Examples include sodium perborate (e.g. mono- or tetra-hydrate), sodium or potassium carbonate peroxyhydrate and equivalent “percarbonate” bleaches, sodium pyrophosphate peroxyhydrate, urea peroxyhydrate, sodium peroxide, sodium persulfate and sodium perphosphate bleach. Inorganic peroxygen bleaches are typically coated with silicate, borate, sulfate or water-soluble surfactants. For example, coated percarbonate particles are available from various commercial sources such as FMC, Solvay Interox, Tokai Denka and Degussa.

Typically, bleach activators are employed such that the molar ratio of bleaching agent to activator ranges from about 1:1 to 12:1, more preferably from about 2:1 to 6:1. The expressed molar ratios assume that the bleach activator is tetraacetylethylenediamine, which possesses two active sites per the reactions described hereinbefore.

One aspect of the invention relates to liquid detergent compositions which are formed from one or more non-aqueous organic solvents in which are suspended particles of inorganic peroxygen bleaching agent, the composite of the invention and optionally a number of other solid insoluble particulate materials. Such non-aqueous compositions generally include one or more surfactants, which serve to enhance the ability of the compositions to keep particulate material suspended and dispersed therein.

The liquid phase of the detergent compositions may comprise one or more non-aqueous organic diluents as the major component. The non-aqueous organic diluents may be either surface active (i.e., non-aqueous surfactant liquids) or non-aqueous, non-surfactant liquids referred to herein as non-aqueous solvents. As used herein, the term “solvent” refers to the non-aqueous liquid portion of the compositions; although some of the components may actually dissolve in the “solvent”-containing liquid portion, other components will be present as particulate material dispersed therein. Thus, the term “solvent” does not require that the solvent material actually dissolves all of the cleaning composition components added thereto.

Further details of suitable non-aqueous surfactant liquids are described in WO 98/00515 and examples include the alkoxylated alcohols, ethylene oxide (EO)-propylene oxide (PO) block polymers, polyhydroxy fatty acid amides, alkylpolysaccharides, and the like. Most preferred of the surfactant liquids are the alcohol alkoxylate nonionic surfactants.

Typically, alcohol alkoxylate nonionic surfactants are preferably present in an amount of from about 1% to about 60% by weight of the composition, more preferably from about 5% to about 50%, even more preferably from about 5% to about 30%.

The amount of total liquid surfactant in the non-aqueous liquid phase will vary depending on the type and nature of other composition components and on the desired composition properties. Typically, the liquid surfactant can comprise from about 15% to about 70% by weight, more preferably from about 20% to about 50% by weight, of the composition.

The liquid phase of the cleaning compositions herein may also comprise one or more non-surfactant, non-aqueous organic solvents, preferably of low polarity. Further details of suitable non-surfactant, non-aqueous organic solvents are described in WO 98/00515 and examples include alkylene glycols, alkylene glycol mono lower alkyl ethers, lower molecular weight polyethylene glycols lower molecular weight methyl esters and amides, and the like.

The non-aqueous, generally low-polarity, non-surfactant organic solvent(s) employed should, of course, be compatible and non-reactive with other composition components, e.g., bleach and/or coated activators, used in the liquid detergent compositions herein. Such a solvent component is preferably utilized in an amount of from about 1% to about 50% by weight, more preferably from about 5% to about 40% by weight, and most preferably from about 10% to about 30% by weight, of the composition.

In one preferred embodiment, the detergent composition of the invention comprises a blend of surfactant and non-surfactant solvents. In systems which employ both non-aqueous surfactant liquids and non-aqueous non-surfactant solvents, the ratio of surfactant to non-surfactant liquids, e.g., the ratio of alcohol alkoxylate to low polarity solvent, within the liquid phase can be used to vary the rheological properties of the detergent compositions eventually formed. Generally, the weight ratio of surfactant liquid to non-surfactant organic solvent will range about 50:1 to 1:50. More preferably, this ratio will range from about 3:1 to 1:3.

Detergent compositions of the present invention may also optionally include anti-redeposition and soil suspension agents, optical brighteners, soil release agents, suds suppressors, enzymes, fabric softening agents, perfumes and colours, as well as other ingredients known to be useful in laundry detergents.

Suitable anti-redeposition and soil-suspension agents include cellulose derivatives such as methylcellulose, carboxymethylcellulose and hydroxyethycellulose, homo- or copolymeric polycarboxylic acids or their salts, such as copolymers of maleic anhydride with ethylene, methylvinyl ether or methacrylic acid. These materials are typically present in amounts of from about 0.5% to about 10% by weight, more preferably from about 1% to about 5% by weight of the composition.

Other useful polymeric materials include polyethylene glycols, particularly those of molecular weight 1000-10000, more particularly 2000 to 8000 and most preferably about 4000. These are typically present in amounts of from about 0.20% to about 5%, more preferably from about 0.25% to about 2.5% by weight. These polymers, along with the above-mentioned homo- or copolymeric polycarboxylate salts are important for improving whiteness maintenance, fabric ash deposition, and cleaning performance on clay, proteinaceous and oxidizable soils in the presence of transition metal impurities.

Soil-release agents useful in compositions of the present invention are conventionally copolymers or terpolymers of terephthalic acid with ethylene glycol and/or propylene glycol units in various arrangements. Examples of such polymers are disclosed in U.S. Pat. No. 4,116,885, U.S. Pat. No. 4,711,730 and EP0272033.

Certain polymeric materials such as polyvinyl pyrrolidones typically of MW 5000-20000, preferably 10000-15000, are also useful in preventing the transfer of labile dyestuffs between fabrics during the washing process.

The detergent composition may further comprise a suds suppressor, such as a silicone or silica/silicone mixture. Examples include alkylated polysiloxane materials, silica aerogels, xerogels and hydrophobic silicas of various types. These materials can be incorporated as particulates in which the suds suppressor is releasably incorporated in a water-soluble or water-dispersible, substantially non-surface-active detergent-impermeable carrier. Alternatively the suds suppressor can be dissolved or dispersed in a liquid carrier and applied by spraying on to one or more of the other components. Further details of suds suppressors and their preferred methods of incorporation are described in WO 94/03568. The suds suppressors described above are typically employed at levels of from about 0.001% to about 0.5% by weight of the composition, preferably from about 0.01% to about 0.1% by weight.

The laundry compositions of the present invention may also comprise one or more enzymes. Preferred enzymatic materials include commercially available amylases, neutral and alkaline proteases, lipases, esterases and cellulases conventionally incorporated into detergent compositions. Suitable examples are disclosed in U.S. Pat. No. 3,519,570.

Fabric softening agents may also be incorporated into laundry compositions in accordance with the present invention. These agents may be inorganic or organic. Inorganic softening agents include smectite clays (as disclosed in GB1400898), whereas organic fabric softening agents include the water insoluble tertiary amines (as disclosed in GB1514276 and EP0011340). Other useful organic fabric softening agents include long chain amides as disclosed in EP0242919. Additional organic ingredients of fabric softening systems include high molecular weight polyethylene oxide materials as disclosed in EP0299575 and EP0313146.

Levels of smectite clay are normally in the range from about 5% to about 15%, more preferably from about 5% to about 10% by weight, with the material being added as a dry mixed component to the remainder of the formulation. Organic fabric softening agents such as the water-insoluble tertiary amines or long chain amide materials are typically incorporated at levels of from about 0.5% to about 5% by weight, more preferably from about 1% to about 3% by weight, whilst the high molecular weight polyethylene oxide materials and the water soluble cationic materials are typically added at levels of from about 0.1% to about 2%, normally from about 0.2% to about 1% by weight of the composition.

In addition, the liquid detergent compositions of the invention may further include thickening agents, such as xanthan gum to control the viscosity and improve perceived quality to the consumer. Thus, the composite suspension is likely to be a consequence of the surfactant action, any self-assembly of the surfactant system to create a gel and the viscosity increasing effect of any thickeners.

A further aspect of the invention relates to a method of preparing a laundry product as described above, said method comprising admixing a composite of the invention with one or more additional conventional laundry composition components.

Yet another aspect of the invention relates to the use of a composite as described above as an additive in a laundry product. Preferably, the laundry product is a detergent, even more preferably, a liquid detergent.

Bleaching System

A further aspect of the invention relates to a bleaching system comprising a composite according to the invention and a bleaching agent. Suitable bleaching agents are as described above.

Alkali Soluble Polymers

Another aspect of the invention relates to an alkali soluble polymer suitable for coating a bleach activating agent, wherein said alkali soluble polymer is as defined above.

The alkali soluble polymers described herein are tailored to optimize the balance of chemical and physical characteristics for the encapsulation of the bleaching agent and the survival of the coated particle in a liquid detergent medium under various ageing regimes. The desired polymers are tailored to be chemically resistant to the detergent medium, film form at the temperatures encountered in the spray coating process, act as an effective physical barrier to the components of the surrounding medium, but demonstrate a tack free coating characteristic even under the most aggressive ageing regimes (for example, at temperatures in excess of 50° C.) to avoid any flocculation and precipitation of the particles. Advantageously, alkali soluble polymers described herein are capable of film forming at modest process temperatures (for example, 50-80° C.).

The present invention is further described by way of the non-limiting examples and with reference to the following Figures, wherein:

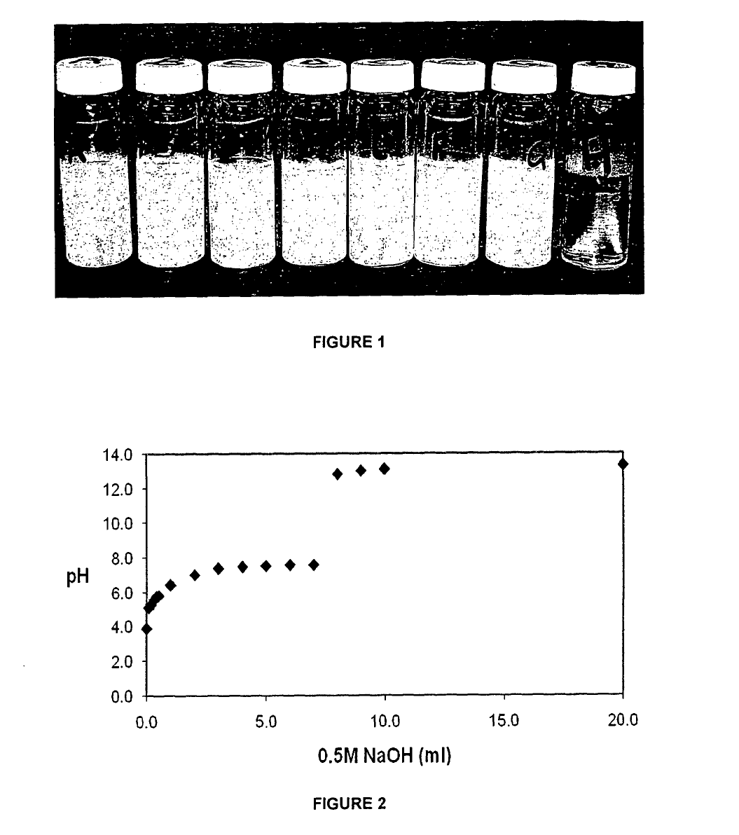

FIG. 1 shows the impact of pH on the polymer NeoCryl BT-26. The sample pH increases from left to right in the pictured samples. At the lowest pH the product is observed as a latex (i.e. the polymer is insoluble), whilst at the highest pH it is a clear solution (i.e. the polymer is soluble).

FIG. 2 shows pKₐ determination for the polymer NeoCryl BT-26.

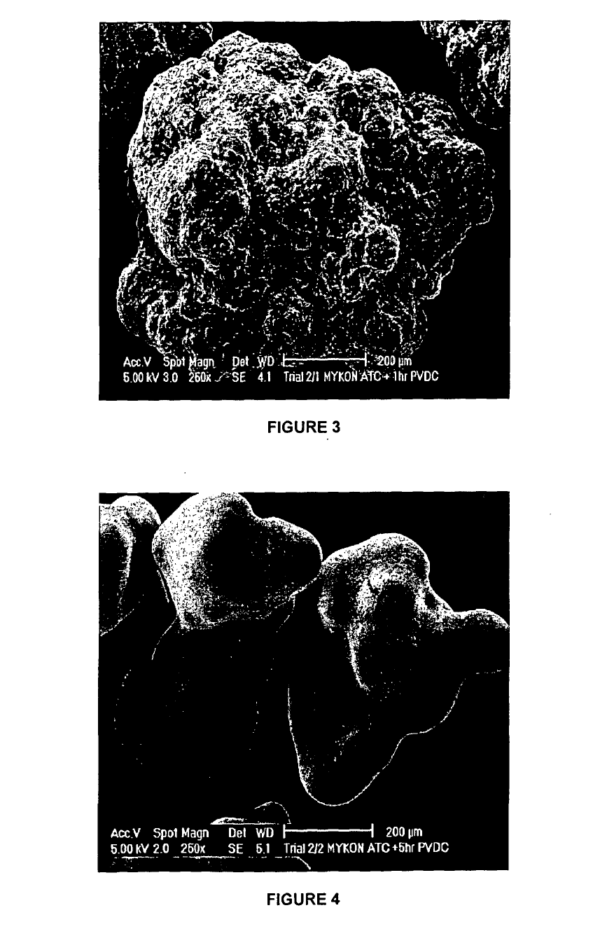

FIG. 3 shows granulated tetraacetylethylenediamine, Mykon ATC (Warwick International Group, Mostyn, UK), spray coated, top spray, with a polymer latex, Ixan Diofan A050, diluted to 20% solids, metered into the fluid bed over one hour to give a composite comprising tetraacetylethylenediamine (70% by weight) and coating (30% by weight). Scanning electron microscopy reveals that an incomplete surface coating has been achieved.

FIG. 4 shows granulated tetraacetylethylenediamine, Mykon ATC, spray coated, top spray, with a polymer latex, Ixan Diofan A050, diluted to 4% solids, metered into the fluid bed over five hours to give a composite comprising tetraacetylethylenediamine (70% by weight) and coating (30% by weight). Scanning electron microscopy confirms that a complete surface coating has been achieved on all the aggregates and effective protection of the bleach activator is achieved.

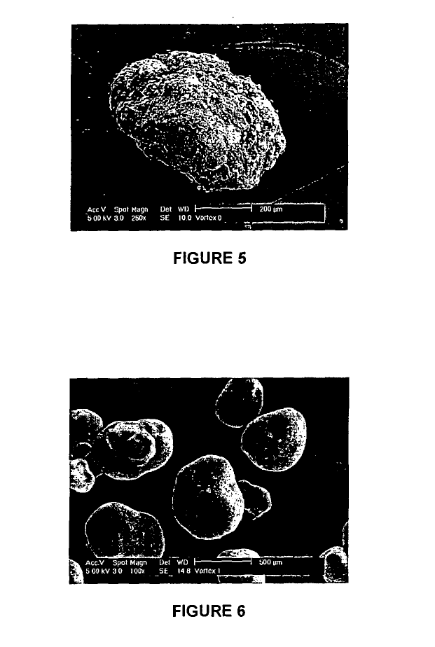

FIG. 5 shows SEM of tetraacetylethylenediamine granulated with an alkali soluble polymer to give an approximately spherical particle ideal for coating with the protecting alkali soluble polymer.

FIG. 6 shows SEM of tetraacetylethylenediamine granulated with an alkali soluble polymer to give an approximately spherical particle and spray coated with a second alkali soluble polymer.

The following examples are intended for illustration only and are not intended to limit the scope of the invention in any way. Specifically, other polymers and bleach activating agents may be used.

### EXAMPLES

In the examples below, reference is made to the following materials:

NeoCryl BT-26, NeoCryl BT-27 and NeoCryl BT-36 were obtained from DSM NeoResins (Waalwijk, The Netherlands);

Ixan Diofan A050 was obtained from Solvin (Brussels, Belgium);

19/JJG/87 is a copolymer of EMA (50.1%), BA (38.6%) and MAA (11.3%);

19/JJG/89 is a copolymer of EMA (55.7%), EHA (33.0%) and MAA (11.3%);

19/JJG/97 is a copolymer of EMA (55.7%), EHA (37.7%) and MAA (6.6%);

19/JJG/111 is a copolymer of EMA (80.0%), EHA (13.4%) and MAA (6.6%);

19/JJG/113 is a copolymer of EMA (77.0%), EHA (16.4%) and MAA (6.6%);

19/JJG/129 is a copolymer of BMA (83.0%), EHA (5.0%) and MAA (12.0%);

19/JJG/143 is a copolymer of EMA (75.4%), EHA (18.0%) and MAA (6.6%);

19/JJG/153 is a copolymer of BMA (55.0%), EHA (33.0%) and MAA (12.0%);

19/JJG/157 is a copolymer of BMA (73.8%), EHA (19.8%) and MAA (6.6%);

19/JJG/161 is a copolymer of BMA (75.0%), EHA (13.0%) and MAA (12.0%);

72/JJG/16 is a copolymer of BMA (83.0%), EHA (5.0%) and MAA (12.0%);

72/JJG/18 is a copolymer of BMA (90.0%), EHA (1.0%) and MAA (9.0%);

72/JJG/24 is a copolymer of BMA (75.0%), EHA (13.0%) and MAA (12.0%);

72/JJG/26 is a copolymer of BMA (75.0%), EHA (13.0%) and MAA (12.0%);

72/JJG/28 is a copolymer of BMA (83.0%), EHA (5.0%) and MAA (12.0%);

72/JJG/30 is a copolymer of BMA (83.0%), EHA (5.0%) and MAA (12.0%);

19/JJG/48 a copolymer of MMA (69.0%), BA (23.0%) and AA (8.0%);

19/JJG/57 a copolymer of MMA (18.7%), BMA (71.3%) and AA (10.0%);

72/JJG/36 a copolymer of STY (50.0%), EHA (38.0%) and MAA (12.0%);

72/JJG/44 a copolymer of STY (60.0%), EHA (31.0%) and MAA (9.0%);

72/JJG/64 a copolymer of STY (33.0%), EHA (43.0%) and MAA (24.0%);

72/JJG/80 a copolymer of STY (50.0%), EHA (35.0%) and MAA (15.0%);

AA (acrylic acid) was obtained from Sigma Aldrich (Gillingham, UK);

MMA (methylmethacrylate) was obtained from Sigma Aldrich (Gillingham, UK);

STY (styrene) was obtained from Sigma Aldrich (Gillingham, UK);

EMA (ethylmethacrylate) was obtained from Sigma Aldrich (Gillingham, UK);

EHA (2-ethylhexylacrylate) was obtained from Sigma Aldrich (Gillingham, UK);

MAA (methacrylic acid) was obtained from Sigma Aldrich (Gillingham, UK);

BA (butylacrylate) was obtained from Sigma Aldrich (Gillingham, UK);

BMA (butylmethacrylate) was obtained from Sigma Aldrich (Gillingham, UK);

Mykon ATC was obtained from Warwick International Group (Mostyn, UK).

Synthesis of an Alkali Soluble Acrylic Copolymer

The alkali soluble acrylic copolymer may be conveniently synthesised by a number of techniques. Methods of interest include emulsion and suspension polymerisation.

Emulsion Polymerisation

To those skilled in the art there are many ways to produce an emulsion polymer. The following procedure was chosen to produce the alkali soluble polymers of this invention.
1. Into a clean, dry, closed jacketed glass vessel charge the bulk of the required water and anionic surfactant.
2. The vessel was fitted with an overhead stirrer, an equalising pressure dropping funnel, a condenser and a thermocouple.
3. The vessel was stirred continuously under a nitrogen blanket and thermostated at 75° C.
4. Separately the required monomers were mixed together and placed in the equalising pressure dropping funnel.
5. Separately a water soluble free radical initiator was dissolved in the balance of the water and added to the stirred glass vessel and the temperature of the bulk aqueous surfactant solution allowed to return to 75° C.
6. After a further 20 minutes the addition of the monomer solution was initiated.
7. The monomer solution was drip fed into the glass vessel over a three hour period.
8. On completion of the monomer addition the stirred vessel was maintained at the set temperature for a further hour.
9. The product was then allowed to cool to ambient and filtered through a 72 microns cloth prior to characterisation and evaluation.
10. The weight percent polymer, particle size distribution and minimum film forming temperature of the resulting latex and the glass transition temperature of the isolated polymer were determined.

Suspension Polymerisation

The alkali soluble acrylic copolymer may also be prepared by an aqueous suspension polymerisation, for example as described in Journal of Applied Polymer Science, 1982, 27, 133-138. The desired mixture of monomers is prepared and suspended, as droplets typically of diameter from 1 micron to 1000 microns, in the water. Preferably stabilisers are added to prevent agglomeration of the droplets. Examples of stabilisers which may be added include polyvinyl alcohol, polyacrylic acid, polyvinyl pyrrolidone, polyalkylene oxide, barium sulphate, magnesium sulphate and sodium sulphate. Agitation of the suspension is preferably employed. The method of agitation employed may help to assist in maintaining the suspension. A free radical initiator commonly serves to initiate polymerisation. The free radical initiator employed is selected according to the types of monomers present. Examples of free radical initiators which may be used to prepare the alkali soluble polymers of the present invention include benzoyl peroxide, dioctanoyl peroxide, 2,2′-azo-bis-isobutyronitrile and 2,2′-azobis(2,4-dimethylvaleronitrile). The selection of a suitable temperature range may be influenced by the nature of the monomers and the initiator present. The polymerisation of the monomers is commonly carried out at solution temperatures ranging from about 150° C. to about 1600° C., preferably from about 50° C. to about 90° C. The polymer beads may be isolated by filtration and optionally washed with water or solvents. The polymer beads may be dissolved in aqueous solution by the use of a neutralising amine such as ammonia, triethyl amine or ethanol amine.

Physical Characteristics of Solid Polymer Sections of NeoCryl BT-26 and BT-27

Solid polymer sections were prepared by casting and drying with sections of each product and mixtures of the products considered. The surface tack and malleability of these dry sections was assessed under ambient laboratory conditions; surface tack as non-tacky or tacky, malleability as soft, pliable, semi-brittle or brittle.

Polymer Composition
(Parts by Weight)

| NeoCryl BT-26 | NeoCryl BT-27 | | |
|---|---|---|---|
| (Tg = 34° C.) | (Tg = 1° C.) | Surface Tack | Malleability |
| 100 | 0 | Non-Tacky | Semi-Brittle |
| 70 | 30 | Non-Tacky | Pliable |
| 50 | 50 | Non-Tacky | Pliable |
| 0 | 100 | Non-Tacky | Soft |

Non-tacky, pliable polymer sections were produced. Naturally such physical characteristics are extremely desirable for the robust and tough coating of the bleach activator.

Water Uptake by Solid Polymer Sections from Acidic Aqueous Media

Solid polymer sections were prepared by casting and drying. The sections were then immersed in acidic aqueous media (25° C./7days). Their weight was found to increase, consistent with their low acid solubility, due to the uptake of a small amount of water from the media and described by the increase in weight (expressed as a percentage of the original section weight).

Water Uptake (%)

| Polymer | Aqueous Medium pH3 | Aqueous Medium pH6 |
|---|---|---|
| NeoCryl BT-26 | 12.0 | 10.0 |
| NeoCryl BT-27 | 12.0 | 13.0 |
| NeoCryl BT-36 | 5.0 | 6.0 |
| 19/JJG/97 | 15.0 | 17.0 |
| 19/JJG/111 | 14.0 | n/a |
| 19/JJG/113 | 14.0 | n/a |

Polymer sections demonstrating low acid solubility and low water uptake were produced.

Solution Characteristics of Alkali Soluble Acrylic Copolymers

The solution characteristics of the alkali soluble acrylic copolymers are critical to the effective protection and release of the bleach activator. The solution characteristics of NeoCryl BT-26 are typical of such materials. Under acid conditions the product is observed as a latex demonstrating a characteristic monomodal particle size distribution with an average hydrodynamic diameter (by photon correlation spectroscopy) of 91.0 nanometers at pH 3.4. When the pH of the latex is raised to values close to the polymer's pKₐ, the polymer becomes progressively more hydrophilic and the droplets swell due to the penetration of water (with an average hydrodynamic diameter by photon correlation spectroscopy of 97.5 nanometers at pH 4.4), but remain largely intact. Finally when the pH is raised to an alkaline value (pH>8.0) the acid-base equilibrium of the carboxylic is shifted to the base (—CO₂⁻) and the polymer becomes fully water soluble; the product is observed to converted from a milky latex to a clear homogeneous viscous solution (see FIG. 1).

The actual pKa of the NeoCryl BT-26 may be simply determined by conducting a titration with aqueous alkali solution. NeoCryl BT-26 (5.0 g) was titrated with 0.5M sodium hydroxide and the pH noted as a function of the alkali addition. A pKa of 7.7 was observed (see FIG. 2).

Spray Coating of Tetraacetylethylenediamine

The spray coating of tetraacetylethylenediamine has been successfully executed with polymers 19/JJG/143, 19/JJG/157 and 19/JJG/161.

Granulation of Tetraacetylethylenediamine

Most conveniently the bleach activator is agglomerated (or granulated) prior to coating to ensure that a particle of the best possible size, shape and physical toughness are realised. In the laboratory, agglomeration is executed using a standard household food blender.

Practically the tetraacetylethylenediamine powder is placed in the blender and mixing commenced. A dilute neutralised solution of the chosen alkali soluble polymer is added progressively until agglomeration was observed (as the formation of regular beads in the bowl of the blender). The agglomerated particles were removed from the blender and allowed to dry prior to classification by sieving. Typically those particles in the size range 200 microns to 400 microns were collected for spray coating.

Spray Coating of Granulated Tetraacetylethylenediamine

The spray coating of granulated tetraacetylethylenediamine has been successfully executed with Ixan Diofan A050 (Solvin, Brussels, Belgium), NeoCryl BT-26, NeoCryl BT-27, NeoCryl BT-36, blends of NeoCryl BT-26 and BT-27 (70:30 and 50:50), blends of BT-27 and BT-36 (30:70 and 50:50), where the Ixan Diofan A050 is a polyvinylidene chloride latex, and alkali soluble acrylic copolymers having the following compositions:

The following alkali soluble polymers have been spray coated onto granulated tetraacetylethylenediamine to give well coated particles, which demonstrate good stability in liquid detergent media:
19/JJG/89
19/JJG/129
19/JJG/143
19/JJG/153
19/JJG/161
72/JJG/16
72/JJG/18
72/JJG/24
72/JJG/26
72/JJG/28
72/JJG/30
72/JJG/36
72/JJG/44
72/JJG/64
72/JJG/80

Granulated Tetraacetylethylenediamine

Granulated tetraacetylethylenediamine, Mykon ATC (Warwick International Group, Mostyn, UK), was spray coated, top spray, with a polymer latex, Ixan Diofan A050, diluted to 20% solids, metered into the fluid bed over one hour. Scanning electron microscopy reveals that an incomplete surface coating has been achieved (see FIG. 3). Such coated materials demonstrate poor stability in acidic hydrogen peroxide containing media.

When the granulated tetraacetylethylenediamine was spray coated, top spray, with Ixan Diofan A050, diluted to 4% solids, metered into the fluid bed over five hours, scanning electron microscopy confirms that a complete surface coating has been achieved on all the aggregates and effective protection of the bleach activator is achieved (see FIG. 4). Such coated materials demonstrate high levels of stability, when placed in acidic hydrogen peroxide containing media.

Retention of Tetraacetylethylenediamine in Liquid Detergent Medium

A granulated tetraacetylethylenediamine was spray coated with NeoCryl BT-26 (pKₐ=7.7) to give a coated product with an average composition of tetraacetylethylenediamine (65 parts by weight), sodium carboxymethylcellulose (5 parts by weight) and polymer (30 parts by weight). The stability of this composite was then assessed by determining the retention of the included tetraacetylethylenediamine on ageing (37° C./48 hours) in a liquid detergent medium, Ace Stain Remover Liquid (Procter & Gamble Company, Cincinnati, USA), whose pH had been adjusted to give media at pH3, pH4 and pH5; i.e. pH values significantly below the pKₐ of the polymer. Poor retention of the tetraacetylethylenediamine was observed; 45% at pH3, 6% at pH4 and 4% at pH5 being retained. This is indicative of the poor barrier created by this commercial polymer. This example illustrates the non-optimum nature of the barrier created by the commercial polymer employed, which is intended for inks and coatings used in graphic art products, rather than the applications covered by the present invention.

pKa of Alkali Soluble Acrylic Copolymers

Alkali soluble acrylic copolymers have been synthesised to give materials with a wide range of desirable pKa values.

| Polymer | pKₐ |
|---|---|
| 19/JJG/123 | 7.5 |
| 72/JJG/14 | 8.0 |
| 19/JJG/131 | 8.5 |

Stability of Composites in Liquid Laundry Detergents

The stability of the composites in commercial liquid laundry detergents was determined. Their behaviour may be compared to that of uncoated tetraacetylethylenediamine and composites produced with Ixan Difan A050. The stability of the tetraacetylethylenediamine and the composite was assessed by determining their gravimetric retention on ageing (40° C./48 hours) in the following liquid detergent media:
1. Fairy Non-Bio Professional Liquid (1), pH8.5;
2. Fairy Non-Bio Liquitabs (1), pH7.8;
3. Fairy Non-Bio Gel (1), pH8.1;
4. Persil Non-Bio Small & Mighty (2), pH8.2;
5. Persil Non-Bio Capsules (2), pH9.5.

(1)—Procter & Gamble Company, Cincinnati, USA.

(2)—Unilever, London, UK.

The following composites were considered:

| Composite | Core | Coating | Core/Coating |
|---|---|---|---|
| T3/R1 | TAED | 19/JJG/157 | 70/30 |
| T3/R5 | TAED | 19/JJG/143 | 60/40 |
| T3/R7 | TAED | 19/JJG/161 | 60/40 |
| T3/R9 | Granulated TAED | Ixan Diofan A050 | 60/40 |

The retention of the tetraacetylethylenediamine, a composite produced with an alkali soluble polymer and the composite produced with PVDC (Ixan Diofan A050) are reported:

Fairy Non-Bio Professional Liquid:

Tetraacetylethylenediamine: 15%.

Composite T3/R5 (with alkali soluble polymer coating): 83%.

Composite T3/R9 (with PVDC coating): 85%.

Fairy Non-Bio Liquitabs:

Tetraacetylethylenediamine: 31%.

Composite T3/R7 (with alkali soluble polymer coating): 72%.

Composite T3/R9 (with PVDC coating): 84%.

Fairy Non-Bio Gel:

Tetraacetylethylenediamine: 39%.

Composite T3/R1 (with alkali soluble polymer coating): 98%.

Composite T3/R9 (with PVDC coating): 84%.

Persil Non-Bio Small & Mighty:

Tetraacetylethylenediamine: 66%.

Composite T3/R1 (with alkali soluble polymer coating): 84%.

Composite T3/R9 (with PVDC coating): 71%.

Persil Non-Bio Capsules:

Tetraacetylethylenediamine: 4%.

Composite T3/R1 (with alkali soluble polymer coating): 80%.

Composite T3/R9 (with PVDC coating): 74%.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

1. A composite comprising:
one or more core units comprising a bleach activating agent; and
an alkali soluble polymer coating on the surface of said one or more core units.

2. A composite according to claim 1 wherein the alkali soluble polymer is insoluble at pH values below its pKa value, and soluble at pH values at or above its pKa value.

3. A composite according to claim 1 wherein the bleach activating agent comprises a precursor containing one or more N-acyl or O-acyl groups,

4. A composite according to claim 3 wherein the bleach activating agent is selected from phenyl acetate, trichloroethyl acetate, mannitol hexaacetate, fructose pentaacetate, benzaldehyde diacetate, sodium p-acetoxybenzene sulphonate, diacetylethylamine, diacetamide, N-formyldiacetamide and [Nu],[Nu]-diacetylaniline, N,N-diacetylethylurethane, triacetylhydrazine, N<1>,N<1>,N<2>-triacetylmethylenediamine, N<1>,N<1>,N<2>,N<2>-tetraacetylethylenediamine (TAED), N<1>,N<1>,N<2>,N<2>-tetraacetylmethylenediamine, N-diacyl derivatives of -semicarbazide, -thiosemicarbazide and -dicyanodiamide, 1,3,4,6-tetraacetyl glycol-uril, 1,3,4,6-tetrapropionyl glycol-uril, sodium 2-acetoxy-5-hexyl-benzene sulphonate, sodium 3,5,5-trimethyl hexanoyloxybenzene sulphonate, sodium 2-ethyl hexanoyloxybenzene sulphonate, sodium nonanoyloxybenzene sulphonate (SNOBS), ethylene glycol diacetate, 2,4-diacetoxy-2,5-dihydrofuran, acetylated sorbitol, acetylated mannitol and mixtures thereof.

5. A composite according to claim 4 wherein the bleach activating agent is TAED or SNOBS.

6. A composite according claim 1 wherein the one or more core units comprise the bleach activating agent in particulate form.

7. A composite according to claim 1 wherein the one or more core units comprise the bleach activating agent in granulate form.

8. A composite according to claim 7 wherein the bleach activating agent is combined with a granulating polymer.

9. A composite according to claim 8 wherein the one or more core units comprise at least about 90% of bleach activating agent and from about 2 to about 10% of said granulating polymer by weight.

10. A composite according to claim 8 wherein the granulating polymer is selected from polyacrylic acid, polyvinyl alcohol, the alkali soluble polymer, an alkali soluble polymer possessing a pKa value equal to or less than that of the coating material, and combinations thereof.

11. A composite according to claim 1 wherein the alkali soluble polymer is prepared from monomers having only one polymerisable double bond.

12. A composite according to claim 1 wherein the alkali soluble polymer is a copolymer prepared from a mixture of at least one dissociating monomer and at least one non-dissociating monomer.

13. A composite according to claim 1 wherein the alkali soluble polymer is of general formula I, 1-[(X)x-(Y)y-(Z)J″-<2> (I) wherein:
R<1> and R<2> are each independently bleach stable polymer end groups;
—(X)x-(Y)y-(Z)z- is a polymer backbone formed from the polymerization of X′, Y and Z′; X′ is a first non-dissociating monomer of formula R<3>R<4>C═CR<5>-CO—OR<6> or R<3>R<4>C═CR<5>R<6>;
V is a second non-dissociating monomer of formula R<3>R<4>C═CR<5>-CO—OR<6> or R<3>RC═CR<5>R<6>;
Z″ is a dissociating monomer of formula R<3>R C=CR<5>-CO—OH or formula R<3>R<4>C═CR<5>-CO-0-(CH2)n-C02H;
each R<3>, R<4> and R<5> is independently hydrogen or an inert aliphatic or aromatic organic moiety;
R<6> is an inert aliphatic or aromatic organic moiety;
x is an integer from 30 to 90;
y is an integer from 0 to 50;
z is an integer from 10 to 60;
wherein the sum of x+y+z=100; and
n is an integer from 2 to 60.

14. A composite according to claim 1 wherein the alkali soluble polymer is an acrylic copolymer formed from monomers selected from methylmethacrylate (MMA), ethylmethacrylate (EMA), butylmethacrylate (BMA), isobutylmethacrylate (iBMA), 2-ethylhexyl methacrylate (EHMA), isobornylmethacrylate (iBoMA), methylacrylate (MA), ethylacrylate (EA), butylacrylate (BA), 2-ethylhexylacrylate (EHA), styrene (STY), acrylic acid (AA), methacrylic acid (MAA) and sodium acrylate (SAA).

15. A composite according to claim 1 wherein the alkali soluble polymer is an acrylic copolymer formed from a mixture of monomers selected from butylmethacrylate (BMA), 2-ethylhexylacrylate (EHA), methacrylic acid (MAA).

16. A composite according to claim 15 wherein the alkali soluble polymer is an acrylic copolymer formed from a mixture of from about 60 to about 90 weight % BMA, from about 1 to about 15 weight % EHA and from about 5 to about 15 weight % MAA.

17. A composite according to claim 1 wherein the alkali soluble polymer is an acrylic copolymer formed from a mixture of monomers selected from styrene (STY), 2-ethylhexylacrylate (EHA), methacrylic acid (MAA).

18. A composite according to claim 17 wherein the alkali soluble polymer is an acrylic copolymer formed from a mixture of from 20 to about 80 weight % STY, from about 20 to about 60 weight % EHA and from about 1 to about 35 weight % MAA.

19. A composite according to claim 1 wherein the alkali soluble polymer is an acrylic copolymer having a molecular weight of from about 20,000 Daltons to 500,000 Daltons.

20. A composite according to claim 1 which comprises from about 10% to about 75%.

21. A process for preparing a composite according to claim 1, said process comprising applying the alkali soluble polymer coating to the surface of said one or more core units.

22. A process according to claim 21 which comprises applying the alkali soluble polymer to said one or more core units by fluid bed coating or fluid bed drying.

23. A process according to claim 21 wherein the alkali soluble polymer is applied to the core units in the form of an alkaline coating solution or an acidic latex.

24. A process according to claim 21 which further comprises the step of preparing the alkali soluble polymer by emulsion polymerisation.

25. A process according to claim 24 which comprises preparing the alkali soluble acrylic copolymer by emulsion polymerisation from a reaction mixture comprising monomers selected from methylmethacrylate (MMA), ethylmethacrylate (EMA), butylmethacrylate (BMA), isobutylmethacrylate (iBA), methyl acrylate (MA), butylacrylate (BA), 2-ethylhexylacrylate (EHA), styrene (STY), acrylic acid (AA), methacrylic acid (MAA) and [beta]-carboxyethylacrylate (BCEA).

26. A process according to claim 25 wherein the reaction mixture is stabilised with an anionic surfactant selected from sodium lauryl sulphate (SLS), sodium benzene alkyl sulphonate (SBAS) and sodium dioctylsulfosuccinate (SDSS).

27. A process according to claim 25 wherein the emulsion polymerisation is initiated with a free radical initiator.

28. A process according to claim 25 wherein the reaction mixture further comprises a chain transfer agent (CTA).

29. A laundry product comprising a composite according to claim 1.

30. A laundry product according to claim 29 which is a liquid laundry product.

31. A laundry product according to claim 30 which is an acidic liquid laundry product.

32. A laundry product according to claim 29 which is a liquid detergent composition.

33. A laundry product according to claim 29 which comprises from about 1% to about 15% of the composite.

34. A laundry product according to claim 29 which further comprises one or more of an anionic surfactant, a non-ionic surfactant, a cationic surfactant, an active oxygen bleaching agent, hydrogen peroxide and water.

35. A laundry product according to claim 29 which further comprises one or more anti-redeposition and soil suspension agents, optical brighteners, soil release agents, suds suppressors, enzymes, fabric softening agents, perfumes and/or colours.

36. A laundry product according to claim 29 which is a powdered laundry product.

37. A method of preparing a laundry product according to claim 29, said method comprising admixing the composite with one or more additional laundry product components.

38. A bleaching system comprising a composite according to claim 1 and a bleaching agent.

39. (canceled)

40. (canceled)

41. A method of generating peracetic acid in situ, said method comprising subjecting a composite according to claim 1 to a pH value at or above the pKa value of the alkali soluble polymer coating in the presence of a bleaching agent.

42. An alkali soluble polymer suitable for coating a bleach activating agent, wherein said alkali soluble polymer is as defined in claim 11.

43. (canceled)

44. (canceled)

45. (canceled)

46. (canceled)

47. (canceled)
